(19) United States
(12) Patent Application Publication
(10) Pub. No.: US 2008/0172074 A1
(43) Pub. Date: Jul. 17, 2008

(54) Transesophageal gastric reduction method and device for practicing same

(75) Inventors: Steve G. Baker, Redmond, WA (US); Sean Totten, Kirkland, WA (US); John C. Bayne, Redmond, WA (US); Clifton A. Alferness, Port Orchard, WA (US); Stefan J. M. Kraemer, Seattle, WA (US)

Correspondence Address: Richard O. Gray, Jr.;Suite. 350, 155-108th Avenue N.E., Bellevue, WA, 98004-5973, US

(73) Assignee: EndoGastric Solutions, Inc.

(21) Appl. No.: 11/654,397

(22) Filed: Jan. 16, 2007

(51) Int. Cl. A61B 17/30

(52) U.S. Cl. 606/157

(57) ABSTRACT

A system and method forms a gastric reduction pouch within a stomach associated with an esophagus. The method includes the steps of delivering, down the esophagus, a substantially planar annular member into the stomach, drawing stomach wall tissue to a juxtaposed relation with the annular member; and securing, to the annular member, the stomach wall tissue juxtaposed to the annular member.

### FIELD OF THE INVENTION

The present invention is generally directed to a therapy for treating obesity. The present invention is more particularly directed to a transesophageal gastric reduction method and device for performing gastric reduction surgery while minimizing surgical invasion.

### BACKGROUND OF THE INVENTION

Obesity is a complex chronic disease involving environment, genetic, physiologic, metabolic, behavioral and psychological components. It is the second leading cause of preventable death in the United States.

Obesity affects nearly one-third of the adult American population (approximately 60 million). The number of overweight and obese Americans has continued to increase since 1960. The trend is not slowing down. Today, 64.5% of adult Americans are categorized as being overweight or obese. Each year, obesity causes at least 300,000 excess deaths in the United States, and healthcare costs of American adults with obesity amounted to approximately $100,000,000,000 (100 billion dollars).

Obesity is not limited to the United States but is increasing worldwide. It is increasing worldwide in both developing and developed countries and is thought to be caused by environmental and behavioral changes resulting from economic development, modernization, and urbanization. Obesity is increasing in children as well. It is believed that the true health consequences of obesity have not yet become totally apparent.

Obesity is currently treated by dietary therapy, physical activity, behavioral therapy, drug therapy, and combinations thereof. Dietary therapy involves instruction on how to adjust a diet to reduce the number of calories eaten. Physical activity strategies include use of aerobic exercise, brisk walking, jogging, cycling, and swimming. Behavioral therapy involves changing diet and physical activity patterns and habits to new behaviors that promote weight loss. Drug therapy is most often used only in conjunction with appropriate lifestyle modifications.

One last treatment for obesity is surgery. Surgery is a treatment option which is generally reserved for persons with severe obesity and those who are morbidly obese. In addition, surgery is not generally performed until other methods of weight loss have been attempted and have been found to be ineffective. Persons who are severely obese are generally unable to physically perform routine daily activities, whether work-related or family functions and have a severely impaired quality of life due to the severity of their obesity.

Most obesity surgeries involve making changes to the stomach and/or small intestines. Currently, there are two types of obesity surgery: (1) restrictive; and (2) combined restrictive and malabsorptive. Operative procedures have been developed for each type of surgery. Each type of surgery has its own risks and side effects.

In restrictive surgery, bands or staples are used to create food intake restriction. The bands or staples are surgically placed near the top of the stomach to section off a portion that is often called a stomach pouch. A small outlet, about the size of a pencil eraser, is left at the bottom of the stomach pouch. Since the outlet is small, food stays in the pouch longer and the feeling of fullness lasts for a longer time. Current operative procedures for restrictive surgery include vertical banded gastroplasty, gastric banding, and laparoscopic adjustable gastric banding. In vertical banded gastroplasty, a stomach pouch is surgically created. In gastric banding, a band is used to create the stomach pouch. In laparoscopic adjustable gastric banding, a less invasive procedure, smaller incisions are made to apply the band. The band is inflatable and may be adjusted over time.

Each of the foregoing therapies for severe obesity has its risks and side effects. Each is invasive surgery and hence exhibits the risks commonly associated with all surgical procedures. Complications may include leaking of stomach juices into the abdomen, injury to the spleen, band slippage, erosion of the stomach by the band, breakdown of the staple line, and stomach pouch stretching from overeating.

However, reductive surgery has proven successful. About 80% of patients lose some weight and 30% reach a normal weight. Hence, the benefits of gastric reduction surgery are generally believed to outweigh the attendant risks and potential complications.

The present invention is directed to an alternative method and device for achieving gastric reduction. As will be seen hereinafter, the method does not require surgical incisions and is thus less invasive than previous reduction therapies.

### SUMMARY OF THE INVENTION

The invention provides a method of forming a gastric reduction pouch within a stomach associated with an esophagus. The method comprises the steps of delivering, down the esophagus, a substantially planar annular member into the stomach, drawing stomach wall tissue to a juxtaposed relation with the annular member, and securing, to the annular member, the stomach wall tissue juxtaposed to the annular member.

The step of securing may include deploying a plurality of fasteners about the annular member. The step of drawing may include forming a fold of stomach tissue juxtaposed to and about the annular member. The step of securing may more particularly include deploying a plurality of fasteners about the annular member to fasten the tissue folds to the annular member. The fold of stomach tissue may be formed oral of the annular member or aboral of the annular member.

The method may further comprise the step of adjusting the circumference of the annular member. The annular member may be star shaped or ring shaped.

The annular member includes a passageway having a size, and the method may further comprise adjusting the size of the passageway. The size of the passageway may be adjusted by inserting a hollow bushing into the annular member passageway. The step of drawing stomach wall tissue to a juxtaposed relation with the annular member may comprise pulling a vacuum in the stomach.

The invention further provides a system for forming a gastric reduction pouch within a stomach associated with an esophagus comprising an annular member and a device that delivers the annular member into the stomach from the esophagus, draws stomach wall tissue to a juxtaposed relation with the annular member, and that secures the juxtaposed stomach wall tissue to the annular member.

In one embodiment, the system comprises a piston member having a distal end and being arranged to be passed down the esophagus to place the distal end within the stomach. The system further comprises an elongated member slidingly arranged on the piston member. The elongated member has a cross-sectional dimension greater than the piston member to form a movable annular support surface. The elongated member further comprises a plurality of fastener deployment channels communicating with the annular support surface. The system further comprises an annular member carried on the piston member adjacent the movable annular support surface, a tissue grabber that grabs stomach tissue and disposes the grabbed stomach tissue between the annular member and the movable annular support surface, and a plurality of fasteners deployable through the fastener deployment channels.

With stomach tissue disposed between the annular member and the movable annular support surface to form a fold of stomach tissue about the piston member between the annular member and the movable annular support surface, the plurality of fasteners may be deployed through the fastener deployment channels to secure the stomach tissue fold to the annular member about the piston member to form the gastric reduction pouch within the stomach.

### BRIEF DESCRIPTION OF THE DRAWINGS

The features of the present invention which are believed to be novel are set forth with particularity in the appended claims. The invention, together with further features and advantages thereof, may best be understood by making reference to the following description taken in conjunction with the accompanying drawings, in the several figures of which like reference numerals identify identical elements, and wherein:

FIG. 1 is a front cross-sectional view of the esophageal-gastro-intestinal tract from a lower portion of the esophagus to the duodenum;

FIG. 2 is a front cross-sectional view with portions cut away illustrating a device according to an embodiment of the invention after being deployed by a method according to one embodiment of the invention and forming a gastric reduction pouch;

FIG. 3 is a front cross-sectional view with portions cut away illustrating another device according to an embodiment of the invention after being deployed and forming a gastric reduction pouch;

FIG. 4 is a front cross-sectional view with portions cut away illustrating the device of FIG. 2 after being deployed by a method according to another embodiment of the invention and forming a gastric reduction pouch;

FIG. 5 is a front cross-sectional view with portions cut away illustrating another device according to an embodiment of the invention after being deployed and forming a gastric reduction pouch;

FIG. 6 is a side plan view illustrating a system for forming a gastric reduction pouch according to the embodiment illustrated in FIG. 4;

FIG. 7 is a side plan view, to an enlarged scale, of a distal portion of the system of FIG. 6;

FIG. 8 is a side plan view illustrating an initial step in the use of the system of FIG. 6 in forming a gastric reduction pouch according to an embodiment of the invention;

FIG. 9 is a side plan view illustrating a further step in the use of the system of FIG. 6;

FIG. 10 is a side plan view illustrating a further step in the use of the system of FIG. 8 wherein the stomach is evacuated to draw the stomach wall to the system;

FIG. 11 is a side plan view illustrating a further step in the use of the system of FIG. 8 wherein the system is folding the stomach wall in juxtaposed relation to a device for maintaining a gastric reduction pouch according to this embodiment of the invention;

FIG. 12 is a side plan view illustrating a still further step in the use of the system of FIG. 8 wherein the system is compacting the fold in the stomach wall juxtaposed to the device for maintaining a gastric reduction pouch according to this embodiment of the invention;

FIG. 13 is a side plan view illustrating a still further step in the use of the system of FIG. 8 wherein the system has secured the folded stomach wall to the device for maintaining a gastric reduction pouch according to this embodiment of the invention;

FIG. 14 is a side plan view illustrating a still further step in the use of the system of FIG. 8 wherein the system is being separated from the device for maintaining a gastric reduction pouch according to this embodiment of the invention;

FIG. 15 is a side plan view of a gastric reduction pouch formed by the system of FIG. 8 being maintained by the device for maintaining the gastric reduction pouch according to this embodiment of the invention;

FIG. 16 is a side view with portions cut away illustrating another system for forming a gastric reduction pouch according to another embodiment of the present invention;

FIG. 17 is a side view with portions cut away illustrating the system of FIG. 16 at an intermediate stage for forming a gastric reduction pouch according to this embodiment of the present invention;

FIG. 18 is a side view with portions cut away illustrating the system of FIG. 16 at a further stage in forming a gastric reduction pouch according to this embodiment of the present invention;

FIG. 19 is a side view with portions cut away illustrating the device deployed by the system of FIGS. 16-18 and the gastric reduction pouch thus formed; and

FIG. 20 is a side view illustrating a manner in which a stomach formed according to the invention may be reduced in size.

### DETAILED DESCRIPTION OF THE INVENTION

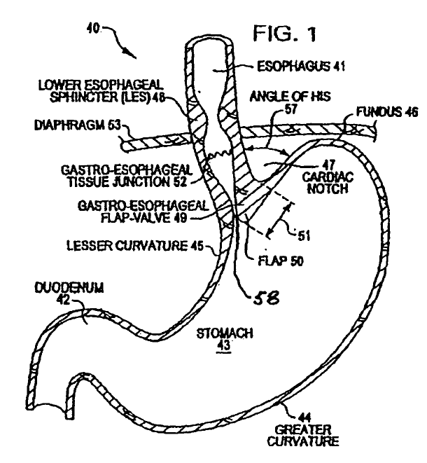

FIG. 1 is a front cross-sectional view of the esophageal-gastro-intestinal tract 40 from a lower portion of the esophagus 41 to the duodenum 42. The stomach 43 is characterized by the greater curvature 44 on the anatomical left side and the lesser curvature 45 on the anatomical right side. The tissue of the outer surfaces of those curvatures is referred to in the art as serosa tissue. As will be seen subsequently, the nature of the serosa tissue is used to advantage for its ability to bond to like serosa tissue. The fundus 46 of the greater curvature 44 forms the superior portion of the stomach 43, and traps gas and air bubbles for burping. The esophageal tract 41 enters the stomach 43 at an esophageal orifice 58 below the superior portion of the fundus 46, forming a cardiac notch 47 and an acute angle with respect to the fundus 46 known as the Angle of His 57. The lower esophageal sphincter (LES) 48 is a discriminating sphincter able to distinguish between burping gas, liquids, and solids, and works in conjunction with the fundus 46 to burp. The gastroesophageal flap valve (GEFV) 49 includes a moveable portion and an opposing more stationary portion. The moveable portion of the GEFV 49 is an approximately 180 degree, semicircular, gastroesophageal flap 50 (alternatively referred to as a “normal moveable flap” or “moveable flap”) formed of tissue at the intersection between the esophagus 41 and the stomach 43. The opposing more stationary portion of the GEFV 49 comprises a portion of the lesser curvature 45 of the stomach 43 adjacent to its junction with the esophagus 41. The gastroesophageal flap 50 of the GEFV 49 principally comprises tissue adjacent to the fundus 46 portion of the stomach 43, is about 4 to 5 cm long (51) at its longest portion, and the length may taper at its anterior and posterior ends. The gastroesophageal flap 50 is partially held against the lesser curvature 45 portion of the stomach 43 by the pressure differential between the stomach 43 and the thorax, and partially by the resiliency and the anatomical structure of the GEFV 49, thus providing the valving function. The GEFV 49 is similar to a flutter valve, with the gastroesophageal flap 50 being flexible and closeable against the other more stationary side.

The esophageal tract is controlled by an upper esophageal sphincter (UES) near the mouth for swallowing, and by the LES 48 and the GEFV 49 at the stomach. The normal anti-reflux barrier is primarily formed by the LES 48 and the GEFV 49 acting in concert to allow food and liquid to enter the stomach, and to considerably resist reflux of stomach contents into the esophagus 48 past the gastroesophageal tissue junction 52. Tissue aboral of the gastroesophageal tissue junction 52 is generally considered part of the stomach because the tissue protected from stomach acid by its own protective mechanisms. Tissue oral of the gastroesophageal junction 52 is generally considered part of the esophagus and it is not protected from injury by prolonged exposure to stomach acid. At the gastroesophageal junction 52, the juncture of the stomach and esophageal tissues form a zigzag line, which is sometimes referred to as the “Z-line.” For the purposes of these specifications, including the claims, “stomach” means the tissue aboral of the gastroesophageal junction 52.

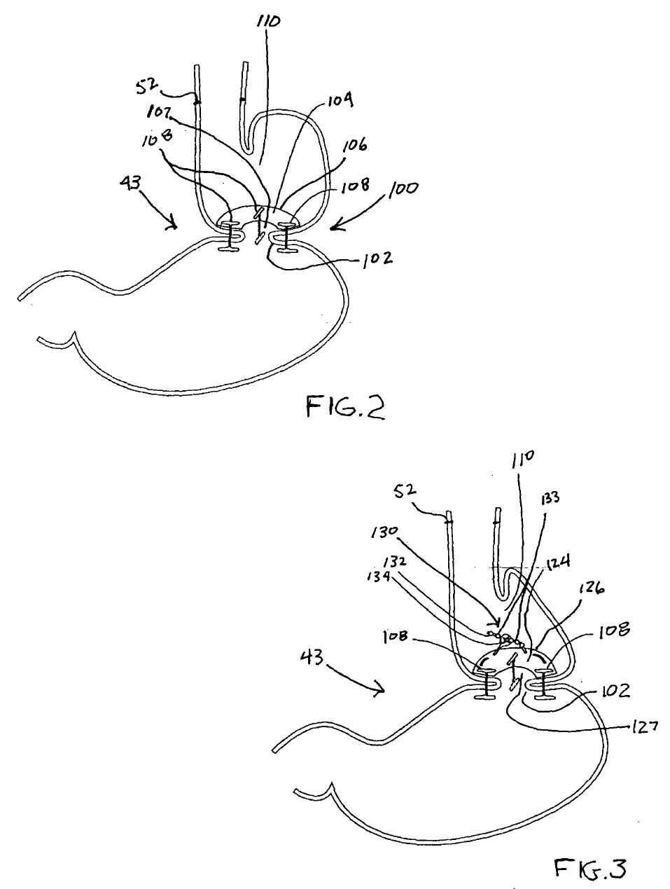

FIGS. 2-4 show various embodiments of gastric reduction according to the present invention. In FIG. 2, it may be noted that a reduced diameter portion 100 of a stomach 43 is formed in accordance with an embodiment of the present invention to result in a gastric reduction pouch 110. The gastric reduction pouch 110 is formed by a fold 102 made circumferentially about the stomach 43 aboral of the Z line 52. The fold 102 is formed by circumferentially gathering the stomach tissue in juxtaposed relation to a major surface 104 of an annular member 106. The annular member 106 is secured to the fold 102 of stomach tissue by a plurality of fasteners 108 that extend through the surface 104 of the annular member 106 and both tissue layers of the fold 102 about the annular member 106.

The annular member 106 may be ring shaped as shown. This provides an opening 107 through which food may pass from the pouch 110 to the rest of the stomach.

The annular member 106 may be formed of most any biocompatible substantially non-elastic material that will maintain its shape. Such materials may include, for example, titanium, Nitinol, silicone rubber, biocompatible plastics, and fabric meshes, of the type and compositions known in the art.

The fasteners may be of the type described in co-pending application Ser. No. 11/121,697, filed Jan. 25, 2005 titled SLITTED TISSUE FIXATION DEVICE AND ASSEMBLIES FOR DEPLOYING THE SAME which application is incorporated herein in its entirety. As may be appreciated, other fasteners and fastener assemblies may be used in securing the stomach tissue fold 102 to the annular member 106 without departing from the present invention. When the annular member is formed of a material that may be pierced by a stylet, the fasteners may be deployed through the annular material. However, if the annular member is formed of a material that may not be pierced by a stylet, apertures may be provided within the major surface 104 through which the fasteners may be deployed.

Referring now to FIG. 3, it shows a similar annular device 126 for maintaining a gastric reduction pouch 110 formed in a stomach 43. As in the previous embodiment, the gastric reduction pouch 110 is formed by a fold 102 made circumferentially about the stomach 43 aboral of the Z line 52. Also as in the previous embodiment, the fold 102 is formed by circumferentially gathering the stomach tissue in juxtaposed relation to a major surface 124 of the annular member 126. The annular member 126 is secured to the fold 102 of stomach tissue by a plurality of fasteners 108 that extend through the surface 104 of the annular member 106 and both tissue layers of the fold 102 about the annular member 106.

The annular member 126 is ring shaped as shown. This provides an opening 127 through which food may pass from the pouch 110 to the rest of the stomach.

The annular member 126 may be seen to further include an adjustment mechanism 130 which may be employed to adjust the size or circumference of the annular member 126. The adjustment mechanism includes a chain 132 which is weaved in and out through the annular member 126 about its circumference, much like a purse string. The chain 132, at one end, includes a series of spaced apart bumps 133, and at the other end, a locking clasp 134. As the chain 132 is pulled through the clasp 134, the circumference of the annular member is made smaller. The co-action of the bumps 133 and clasp 134 maintain the circumference to a desired length. To permit this operation, the annular member 126 must be formed of a substantially flexible material such as, for example, silicone rubber or a fabric mesh.

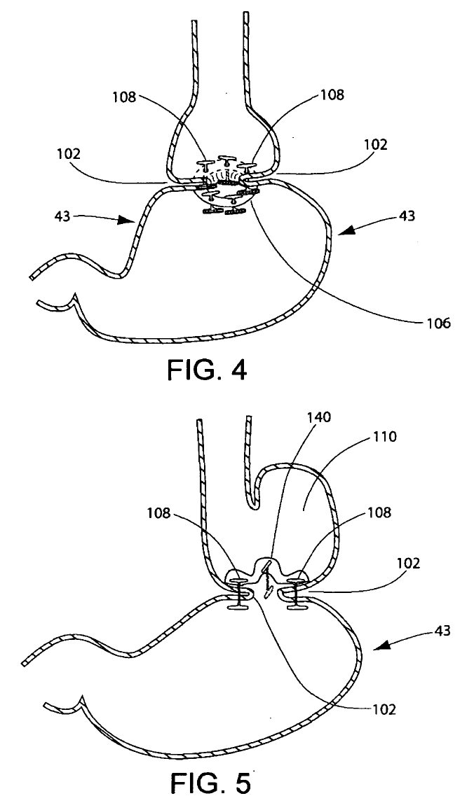

FIG. 4 shows an embodiment where the annular member 106 is positioned on the aboral side of the stomach tissue fold 102. This may be preferable from the standpoint that food passing from the gastric reduction pouch into the rest of the stomach 43 is shielded from the annular member 106 by the fold 102 so that it may be less likely for the food to get caught on the annular member 106. A procedure for positioning the annular member 106 as shown in FIG. 4 is described in detail herein after.

FIG. 5 shows a further embodiment of the invention. Here, the annular member 140 has a sinusoidal star-shaped configuration. This wavy shape of the member 140 serves to increase radial compliance for passing a large bolus of food from the gastric reduction pouch 110 into the rest of the stomach 43. This would even result if the material forming the member 140 is relatively non-compliant. The member may be formed from material such as polypropylene, EPTFE, or PVDF, for example. A plurality of fasteners 108 secure the stomach tissue fold 102 to the annular member 140.

Referring now to FIG. 6, it show a system 200 for forming a gastric reduction pouch in a manner embodying the present invention. The system 200 includes an annular member 206 and a device 202 that draws stomach tissue to the annular member 206 and secures the drawn stomach tissue to the annular member to form a gastric reduction pouch. The device 202 includes an elongated member 204 and an inner sleeve or piston 212. The elongated member is flexible and dimensioned for being fed down an esophagus and into a stomach wherein the gastric reduction pouch is to be formed. The elongated member 204 includes a solid end portion 210 that terminated in a moveable annular support surface 211 at its distal end 208. It also has a central lumen 214, a plurality of fastener guide channels 216, and a plurality of vacuum ports 218. The central lumen 214 is dimensioned to slidingly receive the piston 212.

The piston 212 includes a plurality of vacuum ports 220. It also has a central lumen 222 dimensioned to slidingly receive an endoscope 224.

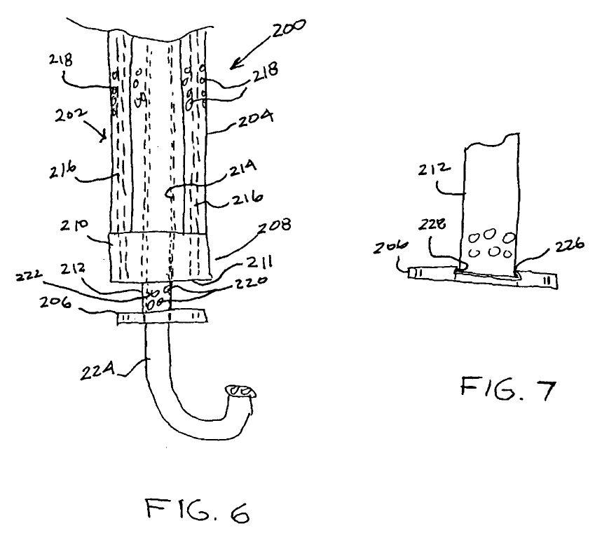

FIG. 7 shows details of the connection between the piston 212 and the annular member 206. As may be noted, the piston 212 terminates in an annular flange 226 which is received within an annular grove 228 of the annular member. Like the elongated member 204, the piston 212 is formed of flexible material. As a result, the annular flange 226 may be pulled out of the annular groove 228 to separate the annular member 206 from the piston 212 after the gastric reduction pouch has been formed.

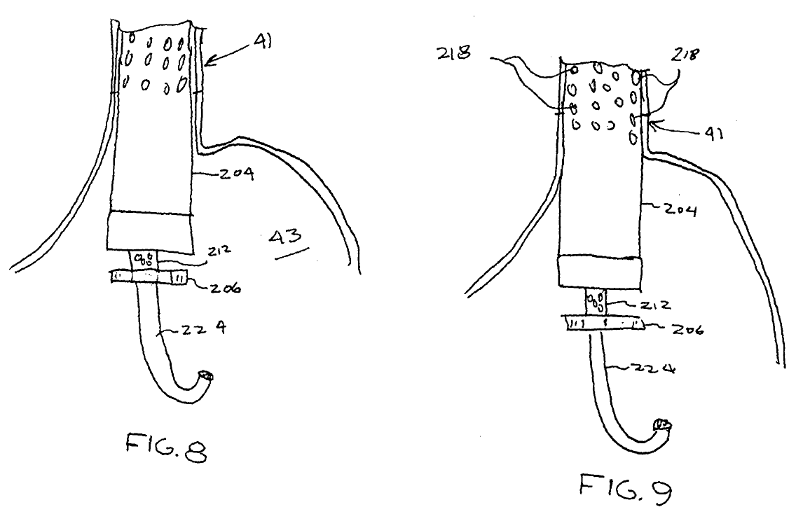

FIG. 8 shows a first step in a procedure for forming a gastric reduction pouch with the system 200 of FIG. 6 according to one embodiment of the invention. Here it may be seen that the elongated member 204 has been fed down the esophagus 41 and into the stomach 43. The endoscope 224 has been advanced through the device and into the stomach 43 to enable visualization.

In the next step of FIG. 9, a vacuum is pulled through the vacuum ports 218 of the elongated member 204. This causes the esophageal wall to be drawn to the elongated member to provide a seal between the esophagus 41 and the elongated 204. In some cases, this step may not be necessary, and hence elective.

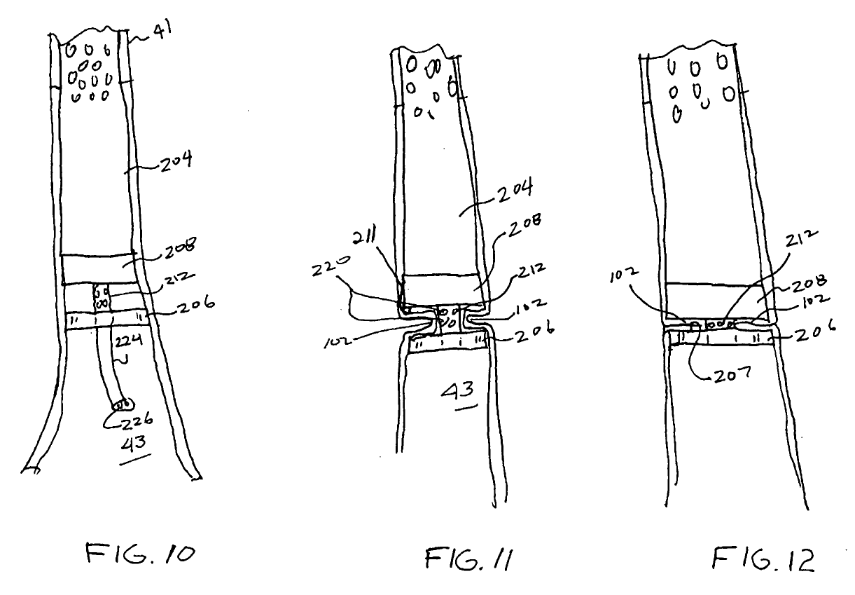

In the next step shown in FIG. 10, a vacuum is pulled through a working channel 226 of the endoscope 224 This causes the wall of the stomach to be drawn to the device and sealed against at least the distal portion 208 of the elongated member 204 and the annular member 206 carried by the sleeve

Next, as shown in FIG. 11, a vacuum is drawn through the vacuum ports 220 of the piston 212 to cause the wall of the stomach to be drawn in between the annular surface 211 and the annular member 206. As will be noted, this causes the stomach wall to be folded inwardly to create a fold 102. Then, as shown in FIG. 12, the piston 212 is moved relative to the elongated member 204 to cause the fold to be squeezed or flattened between the annular member 206 and the distal end portion 208 of the elongated member 204. Hence, the vacuum ports 220 have grasped the stomach tissue and folded it in juxtaposed relation to the major surface 207 of the annular member 206.

The annular member 206 and the stomach tissue fold 102 are now ready to be secured together. To that end, as shown in FIG. 13, a plurality of fasteners 108 may be advanced down the guide channels 216 and deployed through openings 209 of the annular member 206. This may be implemented as described, for example, in the prior referenced application Ser. No. 11/121,697.

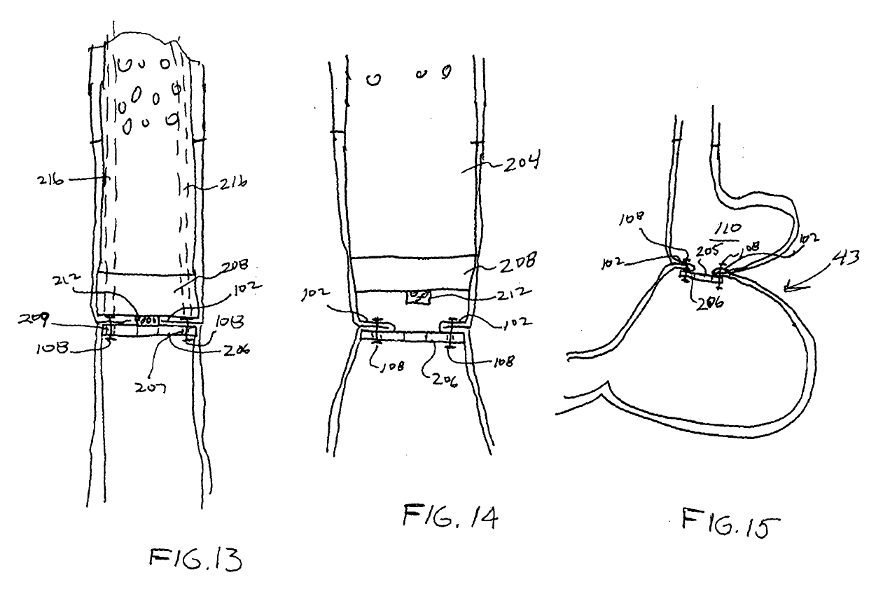

With the stomach tissue fold 102 secured to the annular member 206, the annular member 206 and sleeve 212 are now ready to be separated. This may be accomplished as previously described by the piston 212 being moved relative to the elongated member 204 and thus the annular member 206. The vacuum seals may now be released and the elongated member 204 pulled upward and out through the esophagus. FIG. 14 shows this step being performed.

When the device is fully removed, or as the device is being removed, the stomach may be inflated to assume its new anatomical configuration as shown, for example, in FIG. 15. Here it may be seen that a gastric reduction pouch 110 has been formed in stomach 43. The opening 205 through the annular member 206 together with the fold 102 of stomach tissue define the opening of the gastric reduction pouch into the rest of the stomach 43. As may be noticed, the annular member is disposed on the aboral side of the stomach tissue fold.

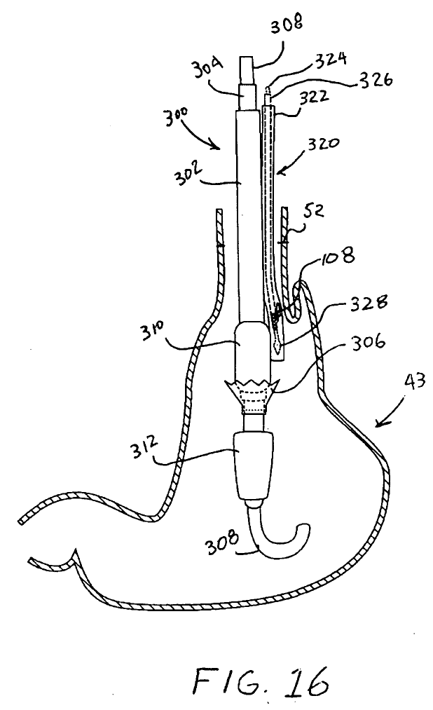

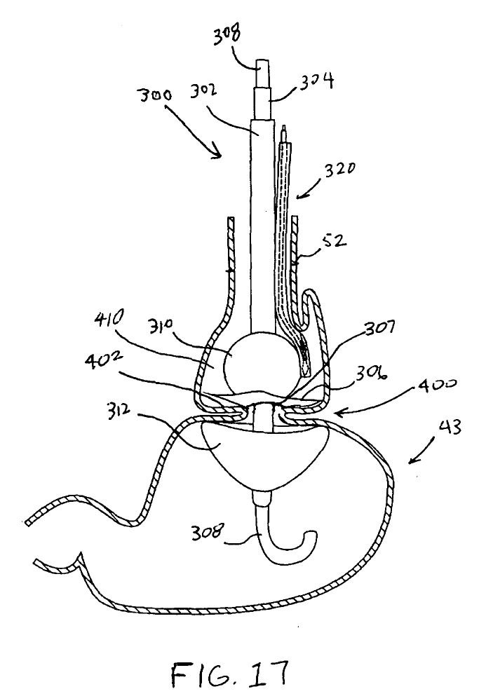

Referring now to FIG. 16, it illustrates a system 300 for deploying an annular member 306 on the oral side of a tissue fold to form a gastric reduction pouch according to another embodiment of the invention. The system 300 generally includes an elongated member 302, an inner tube 304, and an endoscope 308.

At the distal end of the system 300, the elongated member carries a first balloon 310 and the inner tube 304 carries a second balloon 312. Intermediate the first and second balloons 310 and 312 is an annular member 306. The first and second balloons 310 and 312 and the annular member 306 are disposed within the stomach 43 aboral of the Z line 52. The endoscope extends down the inner tube and is retroflexed to provide visualization of the procedure.

The system 300 further includes a fastener deploying device 320. Here the fastener deploying device includes a guide tube that extends along side of the elongated member 302. The fastener deploying divide further includes a stylet 324 the terminates in a sharpened tip 328 and a pusher 326 that pushes a fastener 108 to be deployed along the stylet 324 within the guide tube 322.

In a first stage of deploying the annular member 306, the stomach 43 is evacuated by pulling a vacuum through, for example, a working channel of the endoscope 308. The serves to collapse the tissue of the stomach about the system 300 within the stomach in close proximity to the balloons 310 and 312.

As may be seen in FIG. 17, the balloons 310 and 312 are inflated. The balloon 310 is generally spherical when inflated and the balloon 312 assumes a mushroom shape when inflated. The result of inflating the balloons 310 and 312 is to form a circumferential fold 402 of stomach about the inner tube 304 between the balloons 310 and 312. The inflation of balloon 310 also causes the annular member 306 to open or spread into a substantially planar configuration between the balloon 310 and the tissue fold 402 and in juxtaposed relation to the tissue fold 402.

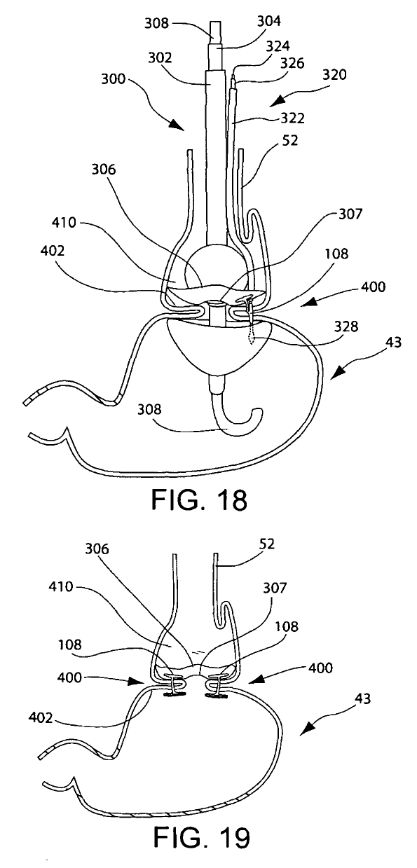

In a further step illustrated in FIG. 18, the fastener deploying device 320 deploys a plurality of fasteners 108 through the tissue fold 402 and the annular member 306, one at a time. Once the fasteners are deployed, the balloons 310 and 312 are deflated. Once collapsed, the balloons 310 and 312 now permit the system 300 to be removed from the stomach 43. During such removal, the second balloon 312 and the inner tube 304 pass through an opening 307 in the annular member 306. The opening 307, as in the previous embodiments permits food to pass to the remainder of the stomach 43 from the pouch 410 thus formed.

FIG. 19 shows the annular member 306 and the formed gastric reduction pouch 410 after the system 300 has been removed from the stomach 43. It may be noted that a reduced diameter portion 400 of a stomach 43 is formed in accordance with this embodiment of the present invention to result in the gastric reduction pouch 410. The gastric reduction pouch 410 is formed by the fold 402 of stomach tissue made circumferentially about the stomach 43 aboral of the Z line 52. The fold 402 is formed in juxtaposed relation to the annular member 306. The annular member 306 is secured to the fold 402 of stomach tissue by a plurality of fasteners 108 that extend through the annular member 306 and both tissue layers of the fold 402 about the opening 307 of the annular member 306. The annular member 306 is disposed on the oral side of the tissue fold 402.

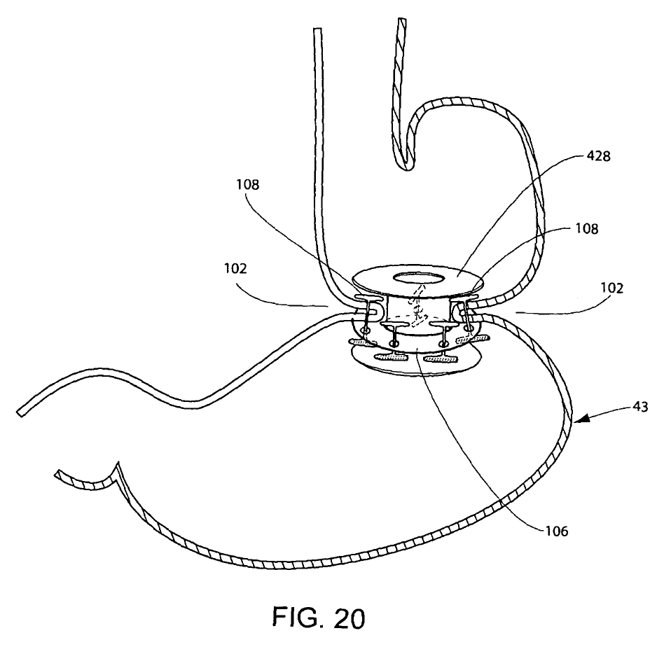

Lastly, FIG. 20 illustrates how the reduced diameter portion 102 of the stomach may be further reduced in size. Here it may be seen that hollow bushing 428 has been inserted into the annular member 106. The bushing may be formed of silicon rubber, for example, to facilitate its being positioned on and within the annular member 106.

While particular embodiments of the present invention have been shown and described, modifications may be made, and it is therefore intended in the appended claims to cover all such changes and modifications which fall within the true spirit and scope of the invention.

What is claimed is:

1. A method of forming a gastric reduction pouch within a stomach associated with an esophagus, comprising:
delivering, down the esophagus, a substantially planar annular member into the stomach;
drawing stomach wall tissue to a juxtaposed relation with the annular member; and
securing, to the annular member, the stomach wall tissue juxtaposed to the annular member.

2. The method of claim 1, wherein the step of securing includes deploying a plurality of fasteners about the annular member.

3. The method of claim 1, wherein the step of drawing includes forming a fold of stomach tissue juxtaposed to and about the annular member.

4. The method of claim 3, wherein the step of securing includes deploying a plurality of fasteners about the annular member to fasten the tissue folds to the annular member.

5. The method of claim 3, wherein the fold of stomach tissue is formed oral of the annular member.

6. The method of claim 3, wherein the fold of stomach tissue is formed aboral of the annular member.

7. The method of claim 1, further comprising the step of adjusting the circumference of the annular member.

8. The method of claim 1, wherein the annular member is star shaped.

9. The method of claim 1, wherein the annular member is ring shaped.

10. The method of claim 1, wherein the annular member includes a passageway having a size, and wherein the method further comprises adjusting the size of the passageway.

11. The method of claim 10 wherein the adjusting step comprises inserting a hollow bushing into the annular member passageway.

12. The method of claim 1, wherein the step of drawing stomach wall tissue to a juxtaposed relation with the annular member comprises pulling a vacuum in the stomach.

13. A system for forming a gastric reduction pouch in a stomach associated with an esophagus, comprising:
a piston member having a distal end, the piston member being arranged to be passed down the esophagus to place the distal end within the stomach;
an elongated member arranged on the piston member, the elongated member having a cross-sectional dimension greater than the piston member to form a movable annular support surface, the elongated member further comprising a plurality of fastener deployment channels communicating with the annular support surface;
an annular member carried on the piston adjacent the movable annular support surface;
a tissue grabber that grabs stomach tissue and disposes the grabbed stomach tissue between the annular member and the movable annular support surface; and
a plurality of fasteners deployable through the fastener deployment channels, whereby,
with stomach tissue disposed between the annular member and the movable annular support surface to form a fold of stomach tissue about the piston between the annular member and the movable annular support surface, the plurality of fasteners may be deployed through the fastener deployment channels to secure the stomach tissue fold to the annular member about the piston to form the gastric reduction pouch within the stomach.

14. The system of claim 13, wherein the tissue grabber comprises a vacuum pulling invaginator.

15. The system of claim 13, wherein the annular member has a passageway forming an opening from the gastric reduction pouch into the stomach, and wherein the system further comprises a hollow bushing arranged to be received within the annular member passageway for reducing the opening from the gastric reduction pouch into the stomach.

16. The system of claim 13, wherein the annular member has a passageway forming an opening from the gastric reduction pouch into the stomach, the opening having a size, and wherein the annular member is adjustable for adjusting the size of the opening from the gastric reduction pouch into the stomach.

17. A system for forming a gastric reduction pouch within a stomach associated with an esophagus, comprising:
an annular member; and
a device that delivers the annular member into the stomach from down the esophagus, draws stomach wall tissue to a juxtaposed relation with the annular member, and that secures the juxtaposed stomach wall tissue to the annular member.

18. The system of claim 17, further comprising a tissue grabber that draws stomach wall tissue under vacuum to the juxtaposed relation with the annular member.

19. The system of claim 17, wherein the annular member has a passageway forming an opening from the gastric reduction pouch into the stomach, and wherein the system further comprises a hollow bushing arranged to be received within the annular member passageway for reducing the opening from the gastric reduction pouch into the stomach.

20. The system of claim 17, wherein the annular member has a passageway forming an opening from the gastric reduction pouch into the stomach, the opening having a size, and wherein the annular member is adjustable for adjusting the size of the opening from the gastric reduction pouch into the stomach.
